(19) United States
(12) Patent Application Publication
(10) Pub. No.: US 2025/0115535 A1
(43) Pub. Date: Apr. 10, 2025

(54) SOLID-STATE SILVER SORBENTS FOR OLEFIN ADSORPTION

(71) Applicant: BOARD OF REGENTS, THE UNIVERSITY OF TEXAS SYSTEM, Austin, TX (US)

(72) Inventor: Rasika Dias, Arlington, TX (US)

(21) Appl. No.: 18/911,658

(22) Filed: Oct. 10, 2024

Related U.S. Application Data
(60) Provisional application No. 63/543,386, filed on Oct. 10, 2023.

(51) Int. Cl. C07C 7/12; C07F 1/00

(52) U.S. Cl. CPC C07C 7/12; C07F 1/005

(57) ABSTRACT

Disclosed herein are compositions for olefin adsorption comprising a trimeric [Ag—R]₃ compound that undergoes a reversible transformation to trimeric [Ag-R·(alkene)]₃ and/or dimeric [Ag-R·(alkene)]₂. The reversible transformation allows one to break adsorbent design trade-offs and achieve low heat of adsorption while retaining high selectivity and uptake. Additionally disclosed is a method to separate alkenes from a mixture.

### STATEMENT OF GOVERNMENT SUPPORT

This invention was made with government support under Grant no. CHE-1954456 awarded by the National Science Foundation. The government has certain rights in the invention.

### BACKGROUND

Effective separation of small gaseous hydrocarbons having comparable sizes and small volatility differences is a challenging endeavor, which is integral to the industrial scale production of high-purity olefins such as ethylene (C₂H₄) and propylene (C₃H₆).¹⁻⁵ It is mainly accomplished by cryogenic distillation under low-temperature and high-pressure conditions, which is capital and energy intensive, and accounts for nearly 0.3% of the global energy consumption just for the purification of ethylene and propylene from their paraffinic counterparts. Alternative methods are clearly desirable to achieve the industrially important olefin-paraffin separation more energy-efficiently and sustainably. Technology based on membranes and porous materials that rely on metal ions such as copper and silver for the selective binding and separation of olefins over paraffins is a promising option and has received significant attention in recent years.

### SUMMARY

Disclosed herein is a group of non-porous, silver complexes supported by highly fluorinated pyrazolates with ideal features for ethylene-ethane separation.^{6, 7} Solution chemistry and single crystal X-ray crystallography show that these molecular silver complexes {[4-(R)-3,5-(CF₃)₂Pz]Ag}₃ ([Ag—R]₃, R=H, Br, CF₃) react with ethylene to form dinuclear silver-ethylene adducts of the type {[4-(R)-3,5-(CF₃)₂Pz]Ag(C₂H₄)}₂([Ag-R·(C₂H₄)]₂). Gas sorption studies revealed that these processes are reversible and can be utilized to separate ethylene very selectively from an ethylene-ethane mixture.

In some aspects, described herein is a composition including an alkene and a compound of Formula I:

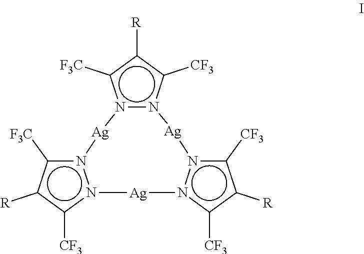

wherein each R is independently chosen from H, CF₃, and Br.

In some aspects, the alkene is ethene, propene, butene, or a mixture thereof.

In some aspects, the composition further includes an alkane, wherein the alkane includes one or more of ethane, propane, and butane.

In some aspects, Formula I exhibits a reversible transformation to Formula II:

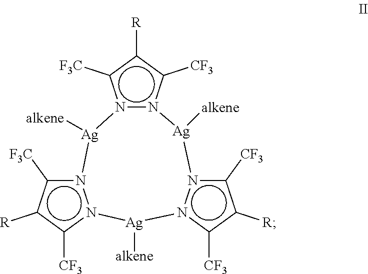

and/or Formula III:

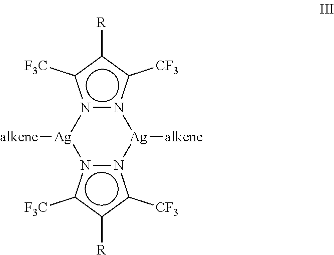

wherein each R is independently chosen from H, CF₃, and Br, and wherein the alkene of the composition is a same alkene of Formula II and Formula III.

In some aspects, Formula II is formed by an alkene adsorption on Formula I when R is H.

In some aspects, Formula III is formed by an alkene adsorption on Formula I when R is CF₃, or Br.

In some aspects, an alkene desorption transforms Formula II to Formula I.

In some aspects, an alkene desorption transforms Formula III to Formula I.

In some aspects, the reversible transformation is an exothermic structural rearrangement of Formula 1.

In some aspects, the reversible transformation is a solid-state transformation.

In some aspects, described herein is a method of separating an alkene from a mixture, the method including: contacting the mixture to a compound having Formula I to form a complex having Formula II and/or Formula III; wherein each R is independently chosen from H, CF₃, and Br.

In some aspects, the mixture is contacted with the compound having Formula I at a pressure below a partial pressure of the alkene.

In some aspects, the mixture is contacted with the compound having Formula I at a temperature from −75° C. to 25° C.

In some aspects, the mixture is contacted with the compound having Formula I at a pressure from ambient pressure to 100 kPa.

In some aspects, the mixture is contacted with the compound having Formula I at a pressure from 100 kPa to 6,000 kPa.

In some aspects, the mixture is contacted with the compound having Formula I at a pressure from 300 kPa to 1,000 kPa.

In some aspects, the method further includes reducing pressure to ambient pressure or below after forming the complex having Formula II and/or Formula III and collecting the alkene.

In some aspects, the method further includes increasing temperature after forming the complex having Formula II and/or Formula III and collecting the alkene.

In some aspects, the mixture is contacted with the compound having Formula I in the presence of a solvent.

In some aspects, the mixture is a hydrocarbon feed gas mixture.

### BRIEF DESCRIPTION OF DRAWINGS

FIG. 1 shows trinuclear silver(I)-pyrazolates utilized in this work, {[4-R-3,5-(CF₃)₂Pz]Ag}₃ ([Ag—R]₃, R=H, Br, CF₃).

FIG. 2 shows the molecular structure of [Ag—CF₃]₃·CH₂C₂ (top) and [Ag-CF₃·(C₂H₄)]₂ (bottom) obtained from solution process and single crystal X-ray diffraction studies.

FIG. 3 shows (upper) the conversion of [Ag—CF₃]₃ to [Ag-CF₃·(C₂H₄)]₂ occurs at 3-5 bar at 295 K as evident from this experiment from 5-60-5 bar of ethylene at 295 K; and (lower) the [Ag-CF₃·(C₂H₄)]₂ to [Ag—CF₃]₃ conversion (the ethylene loss) under helium flow (˜1 bar) at 295 K.

FIG. 4 shows the molecular structure of [Ag-CF₃·(C₂H₄)]₂ obtained from solution (top) using single crystal X-ray diffraction studies and solid-state chemistry (bottom) from powder X-ray diffraction.

FIG. 5 shows the molecular structure of [Ag—CF₃]₃ obtained by in situ powder X-ray diffraction studies of the materials from solid-gas chemistry.

FIG. 6 shows the evolution of the in situ PXRD patterns showing of ethylene and solid [Ag—H]₃ reaction at 295 K with increasing ethylene pressure. No reaction was observed at 60 bar.

FIG. 7 shows (upper) the evolution of the in situ PXRD patterns showing of ethylene and solid [Ag—H]₃ reaction at 10 bar ethylene with decreasing temperature. Formation of a new phase confirmed as {[3,5-(CF₃)₂Pz]Ag(C₂H₄)}₃ ([Ag-H·(C₂H₄)]₃) was observed starting at 223K; and (lower) the evolution of the in situ PXRD patterns showing decomposition of {[3,5-(CF₃)₂Pz]Ag(C₂H₄)}₃ ([Ag-H·(C₂H₄)]₃) to produce ethylene free starting phase [Ag—H]₃ at 10 bar ethylene with increasing temperature. Conversion to [Ag—H]₃ was observed at 262K.

FIG. 8 shows (upper) the evolution of the in situ PXRD patterns showing of ethylene and solid [Ag—H]₃ reaction at 5 bar ethylene with decreasing temperature and formation of a new phase was observed starting 206K, {[3,5-(CF₃)₂Pz]Ag(C₂H₄)}₃ ([Ag-H·(C₂H₄)]₃); and (lower) Evolution of the in situ PXRD patterns showing decomposition of {[3,5-(CF₃)₂Pz]Ag(C₂H₄)}₃ ([Ag-H·(C₂H₄)]₃) to produce ethylene free starting phase [Ag—H]₃ at 5 bar ethylene with increasing temperature. Conversion to [Ag—H]₃ was observed at 256K.

FIG. 9 shows in situ PXRD based molecular structure of the silver-ethylene [Ag-H·(C₂H₄)]₃ intermediate generated by in situ solid-gas chemistry (top); and selected atoms showing only the Ag₃N₆(C₂H₄)₃ moiety and the distorted Ag₃N₆ core (bottom).

FIG. 10 shows two views of [Ag-H·(C₂H₄)]₃ molecular structure, determined from powder diffraction data. CCDC deposition 2266818.

FIG. 11 shows evolution from the two-phase [Ag—H]₃ starting material in the bottom panel, to a disordered [Ag—H]₃ trimer phase (generated after ethylene uptake and removal cycle) in the middle panel. After 12 hours, the sample had settled into a clean mixture of the original two phases.

FIG. 12 shows (top) [Ag—H]₃ from room temperature to 110K at 45 bar of ethylene; (bottom) warming [Ag-H·(C₂H₄)]₃ while maintaining very high ethylene pressure of 70 bar. In both cases no signs of a new phase (potentially [Ag-H·(C₂H₄)]₂) were observed. Warming leads to ethylene loss and [Ag—H]₃ formation, even under high ethylene pressure.

FIG. 13 shows (upper) the evolution of the in situ PXRD patterns showing of ethylene and solid [Ag—Br]₃ reaction at 10 bar ethylene with decreasing temperature. Formation of a new phase confirmed as {[4-Br-3,5-(CF₃)₂Pz]Ag(C₂H₄)}₂ ([Ag-Br·(C₂H₄)]₂) was observed starting at 220K; and (lower) the evolution of the in situ PXRD patterns showing decomposition of {[4-Br-3,5-(CF₃)₂Pz]Ag(C₂H₄)}₂ ([Ag-Br·(C₂H₄)]₂) to produce ethylene free starting phase [Ag—Br]₃ at 10 bar ethylene with increasing temperature. Conversion to [Ag—Br]₃ was observed at 295K, which was slow at 295K and 10 bar ethylene. Single crystal XRD structure of [Ag—Br]₃ (crystallized from solutions) has been reported and has CCDC ref code PIVJUB.¹³

FIG. 14 shows (upper) the reaction of ethylene with [Ag—Br]₃ to form {[4-Br-3,5-(CF₃)₂Pz]Ag(C₂H₄)}₂ ([Ag-Br·(C₂H₄)]₂); and (lower) the decomposition of {[4-Br-3,5-(CF₃)₂Pz]Ag(C₂H₄)}₂ ([Ag-Br·(C₂H₄)]₂) to produce ethylene free starting phase [Ag—Br]₃ with the release of ethylene.

FIG. 15 shows the molecular structure of in situ generated [Ag-Br·(C₂H₄)]₂ based on powder X-ray diffraction data.

FIG. 16 shows two views of molecular structure of [Ag-Br·(C₂H₄)]₂ based on above in situ powder X-ray diffraction data.

FIG. 17 shows Gibbs free energy diagram for the proposed mechanism for dimer formation, involving [Ag—H]₃, [Ag—Br]₃, and [Ag—CF₃]₃ at 298K. Values given per [Ag—R]₃ unit in kcal/mol (R=H, Br, or CF₃).

FIG. 18 shows graphical representation of Gibbs free energy difference for reaction coordinates at different temperatures.

FIG. 19 shows ¹⁹F NMR spectra of [Ag-CF₃·(C₂H₄)]₂/[Ag—CF₃]₃/ethylene at various temperatures. (Referenced to internal standard at constant value, 1,3,5-(CF₃)₃C₆H₃ was used as the internal standard (−63.41 ppm)). The data were collected using crystalline [Ag-CF₃·(C₂H₄)]₂.

FIG. 20 shows crystal data and structure refinement for [Ag-CF₃]₃·CH₂Cl₂.

FIG. 21 shows crystal data and structure refinement for [Ag-CF₃·(C₂H₄)]₂.

### DETAILED SPECIFICATION

The present invention can be understood more readily by reference to the following detailed description, examples, drawings, and claims, and their previous and following description. However, before the present compositions, articles, devices, systems, and/or methods are disclosed and described, it is to be understood that this invention is not limited to the specific compositions, articles, devices, systems, and/or methods disclosed unless otherwise specified, as such can, of course, vary. It is also to be understood that the terminology used herein is for the purpose of describing particular aspects only and is not intended to be limiting.

The following description of the invention is also provided as an enabling teaching of the invention in its best, currently known aspect. To this end, those of ordinary skill in the relevant art will recognize and appreciate that changes and modifications can be made to the various aspects of the invention described herein, while still obtaining the beneficial results of the present invention. It will also be apparent that some of the desired benefits of the present invention can be obtained by selecting some of the features of the present invention without utilizing other features. Accordingly, those of ordinary skill in the relevant art will recognize that many modifications and adaptations to the present invention are possible and can even be desirable in certain circumstances and are thus also a part of the present invention. Thus, the following description is provided as illustrative of the principles of the present invention and not in limitation thereof.

### Definitions

Various combinations of elements of this disclosure are encompassed by this invention, e.g. combinations of elements from dependent claims that depend upon the same independent claim.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification

All publications mentioned herein are incorporated herein by reference to disclose and describe the methods and/or materials in connection with which the publications are cited.

It is also to be understood that the terminology used herein is for the purpose of describing particular aspects only and is not intended to be limiting. As used in the specification and in the claims, the term “comprising” may include the aspects “consisting of” and “consisting essentially of” It is further understood that the term “comprise” means “include,” “have,” and “contain,” but is not limited to, and is not intended to exclude, for example, other additives, components, integers, or steps.

Unless defined otherwise, all technical and scientific terms used herein have the same meaning as commonly understood by one of ordinary skill in the art to which this invention belongs. In this specification and in the claims which follow, reference will be made to a number of terms which shall be defined herein.

As used herein, the singular forms “a,” “an” and “the” include plural referents unless the context clearly dictates otherwise. Thus, for example, reference to a “complex” or “a composition” includes aspects having two or more complexes or compositions unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. By “about” is meant within 5% of the value, e.g. within 4, 3, 2, or 1% of the value. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur, and that the description includes instances where said event or circumstance occurs and instances where it does not.

As used herein, the term “substantially,” in, for example, the context “substantially free” refers to a composition having less than about 1% by weight of the stated component. This can include, for example, aspects of less than about 0.5% by weight, less than about 0.1% by weight, less than about 0.05% by weight, or less than about 0.01% by weight of the stated material, based on the total weight of the composition.

It is further understood that the term “substantially,” when used in reference to an amount of composition or a component in a composition, refers at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature or component.

As used herein, the term “substantially,” in, for example, the context “substantially identical reference composition” or “substantially identical reference process” refers to a reference composition comprising substantially identical components, or process steps, in the absence of an inventive component. In another exemplary aspect, the term “substantially,” in, for example, the context “substantially identical reference composition,” refers to a reference composition comprising substantially identical components and wherein an inventive component is substituted with a common or conventionally known in the art component. For example, a substantially identical reference composition as described herein can comprise a substantially identical composition comprising an alkene and a compound having the formula: [3,4,5-tris(trifluoromethyl)pyrazolyl)Ag]₃, ([(3,4,5-(CF₃)₃Pz)Ag]₃, [Ag—CF₃]₃).

As used herein, the term “‘step’ pressure,” refers to a pressure value where a steep change in pressure as shown in gas adsorption isotherm occurs. It is understood that the step pressure is been achieved through phase changes induced by osmotic pressure and chemical interaction and is facilitated by flexible ligands, rearrangement of coordination spheres, or spin-crossover. Alternatively, the step pressure can be achieved through intermolecular interactions inducing ordering of the gas (adsorbate) phase such as self-propagating reactions and clathrate formation. It is further understood that the ‘step’ pressure is dependent on a system and can be determined from the system's gas adsorption isotherm.

While aspects of the present invention can be described and claimed in a particular statutory class, such as the system statutory class, this is for convenience only, and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The present disclosure may be understood more readily by reference to the following detailed description of various aspects and the examples included therein and to the Figures and their previous and following description.

Due to the current interests and importance of group 11 metals in multiple areas of olefin chemistry, disclosed herein is a group of non-porous, silver complexes supported by highly fluorinated pyrazolates with ideal features for ethylene-ethane separation.^{6, 7} Solution chemistry and single crystal X-ray crystallography show that these molecular silver(I) complexes {[4-(R)-3,5-(CF₃)₂Pz]Ag}₃ ([Ag—R]₃, R=H, Br, CF₃) react with ethylene to form dinuclear silver-ethylene adducts of the type {[4-(R)-3,5-(CF₃)₂Pz]Ag(C₂H₄)}₂ ([Ag-R·(C₂H₄)]₂, R=CF₃, Br) and trinuclear silver-ethylene adducts of the type {[4-(R)-3,5-(CF₃)₂Pz]Ag(C₂H₄)}₃ ([Ag—R·(C₂H₄)]₃, R=H). Gas sorption studies revealed that these processes are reversible and can be utilized to separate ethylene selectively from an ethylene-ethane mixture.

### Compositions

In certain aspects, the present disclosure relates to a compound having Formula I:

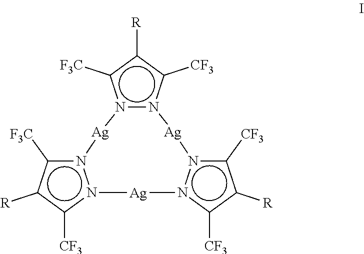

wherein each R is independently chosen from H, CF₃, and Br. For example, R can be chosen from CF₃ and Br. In another example, R can be H.

In other aspects, the present disclosure relates to a composition comprising an alkene and the compound of Formula I. The alkene can be ethene, propene, 1-butene, 2-butene or mixtures thereof. Further the composition can comprise an alkane. The alkanes can be ethane, propane, butane or mixtures thereof.

In further aspects, disclosed herein is a complex having Formula II:

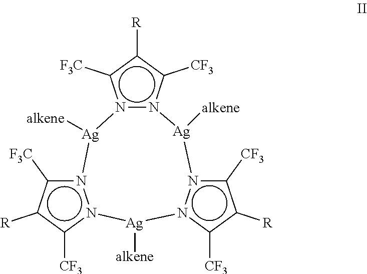

wherein each R is independently chosen from H, CF₃, and Br. In an example, Formula I exhibits a reversible transformation to Formula II when R is H.

In still other aspects, alkene can be ethene, propene, 1-butene, 2-butene, or mixtures thereof. Without wishing to be bound by theory, the complexes of Formula II can be present with compounds of Formula I and an alkene, and such compositions are expressly contemplated and disclosed herein.

In yet further aspects, disclosed herein is a complex having Formula III:

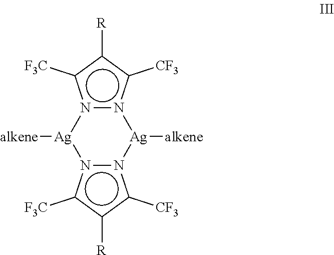

wherein each R is independently chosen from H, CF₃, and Br. In an example, Formula I exhibits a reversible transformation to Formula III when R is CF₃, or Br.

In still other aspects, alkene can be ethene, propene, 1-butene, 2-butene, or mixtures thereof. Without wishing to be bound by theory, the complexes of Formula III can be present with the compound of Formula I and an alkene, and such compositions are expressly contemplated and disclosed herein.

In still other aspects, the complexes of Formula II can be present with the complex of Formula III, compounds of Formula I and an alkene, and such compositions are expressly contemplated and disclosed herein.

It should be noted that while Formula II and Formula III show a solid line between a silver atom and “alkene”, this is not meant to imply a single bond. It is meant merely to illustrate a coordination of the silver to the alkene.

In yet further aspects, the compound having Formula I is in its solid state. Yet in other examples, the composition further comprises a solvent. Examples of suitable solvents include methylene chloride and chloroform.

In still further aspects, disclosed herein are adsorption materials comprising the composition of Formula I and/or Formula II and a substrate. In still further aspects, disclosed herein are adsorption materials comprising the composition of Formula I and/or Formula III and a substrate. The substrate can be a bead, film, particle, or membrane, which can be made of either an inorganic or polymeric substrate. In some examples, the adsorption materials can be in a fixed and/or fluidized bed, e.g., in a fixed and/or fluidized bed temperature and/or pressure swing adsorption process.

### Methods

In certain aspects, the present disclosure relates to a method of separating an alkene from a mixture. In some aspects, the method comprises contacting the mixture with the compound having Formula I to form a complex having Formula II and/or Formula III, wherein the alkene moiety in the complex is the alkene being separated from the mixture. In some examples, the mixture can be contacted with the compound having Formula I at high pressure and ambient temperature or at low temperatures and ambient pressure. In specific examples, contacting the compound having Formula I with the mixture can occur at pressures at or above ambient pressure.

The alkene can be ethene, propene, 1-butene, 2-butene, or a mixture thereof. While not wishing to be bound by theory, the alkene can be separated from any gas that does not contain a carbon-carbon double bond and/or pi electrons that would interact with the compounds having Formula I. For example, alkenes can be separated from N₂, methane, carbon dioxide.

In still further aspects, the mixture is a hydrocarbon feed gas. The hydrocarbon feed gas can be obtained by any methods known in the art. In still further aspects, the hydrocarbon feed gas mixture can comprise one or more alkenes and one or more alkanes. In yet other aspects, the hydrocarbon feed gas mixture can comprise ethylene, propylene, butene, or any combination thereof. It is understood that the hydrocarbon feed gas can be a raw olefin gas or a pretreated olefin gas. In aspects where the hydrocarbon feed gas is a pretreated olefin gas, the gas can be pretreated to remove one or more components depending on the desired application. In some exemplary aspects, the one or more alkenes present in the hydrocarbon feed gas mixture can comprise ethylene. In yet other aspects, the one or more alkanes present in the hydrocarbon feed gas mixture can comprise ethane. In still further exemplary aspects, the disclosed compositions can be used to separate ethylene from ethane. However, it is understood that such aspects are exemplary and non-limiting and the complexes described herein can be also used to separate other alkenes from other alkanes. It is further understood that the processes and the complexes described herein can be used to separate, for example, propylene gas from other components of the hydrocarbon feed gas. It is further understood that in some exemplary aspects, the presence of ethylene in the hydrocarbon feed gas mixture is not required in order to efficiently separate other alkenes from various alkanes. It is understood that in aspects, where ethylene is not present, other alkenes, such as propylene, or butene can adsorb similarly on a trinuclear silver(I) pyrazolate complex to form a specific dinuclear silver(I)-alkene complex and/or trinuclear silver(I)-alkene complex.

The mixture can be contacted with the composition having Formula I at a temperature from −75° C. to 25° C. In some specific examples, the mixture can be contacted with the composition having Formula I at a temperature from −75° C. to −50° C., from −75° C. to −25° C., from −75° C. to 0° C., from −50° C. to −25° C., from −50° C. to 0° C., from −50° C. to 25° C., from −25° C. to 0° C., from −25° C. to 25° C., from 0° C. to 25° C. In further examples, the mixture can be contacted with the composition having Formula I at −70, −60, −50, −40, −30, −20, −10, 0, 10, or 20° C., where any of the stated values can form an upper or lower endpoint of a range.

In still other examples, the mixture can be contacted with the composition of Formula I at pressures from ambient pressure to 100 kPa. In still other examples, the mixture can be contacted with the composition of Formula I at pressures from 100 kPa to 6,000 kPa, e.g., from 300 kPa to 1000 kPa. In specific examples the pressure can be 100 kPa, 200 kPa, 300 kPa, 400 kPa, 500 kPa, 600 kPa, 700 kPa, 800 kPa, 900 kPa, 1000 kPa, 2000 kPa, 3000 kPa, 4,000 kPa, 5,000 kPa, or 6,000 kPa, where any of the stated values can form an upper or lower endpoint of a range.

In other examples, the pressure can be reduced to ambient pressure or below after forming the complex having Formula II and/or Formula III and the alkene can be isolated or recovered. In other examples, the temperature can be increased after forming the complex having Formula II and/or Formula III and the alkene can be isolated or recovered. Still further, the pressure and temperature can be adjusted to conditions that result in the release of the alkene from the complex having Formula II and/or Formula III and the alkene can then be isolated or recovered.

In still further examples, the method can be a solid-state method wherein the compound having Formula I is in its solid state when contacted with the alkene. Yet in other examples, the compound having Formula I can be contacted with the alkene in the presence of a solvent. Examples of suitable solvents include methylene chloride and chloroform.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Solid-gas reactions and in situ powder X-ray diffraction investigations of trinuclear silver complexes {[3,4,5-(CF₃)₃Pz]Ag}₃ and {[4-Br-3,5-(CF₃)₂Pz]Ag}₃ supported by highly fluorinated pyrazolates reveal that they undergo intricate ethylene-triggered structural transformations in the solid-state producing dinuclear silver-ethylene adducts. Despite the complexity, the chemistry is reversible producing precursor trimers with the loss of ethylene. Less reactive {[3,5-(CF₃)₂Pz]Ag}₃ under ethylene pressure and low-temperature conditions stops at an unusual silver-ethylene complex in the trinuclear state, which could serve as a model for intermediates likely present in more common trimer-dimer reorganizations described above. Complete structural data of three novel silver-ethylene complexes are presented together with a thorough computational analysis of the mechanism.

### INTRODUCTION

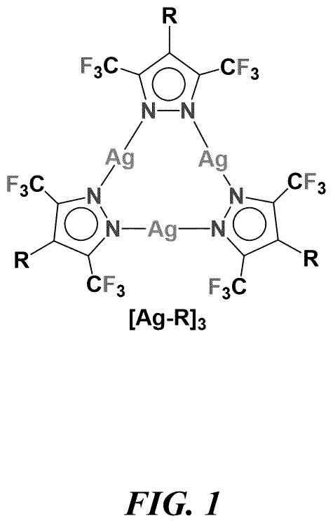

Trinuclear silver(I) complexes of fluorinated pyrazolates have attracted significant interest because many of them show interesting p-acid properties, luminescence, argentophilic contacts, and useful applications.⁸⁻¹⁵ For example, {[3,5-(CF₃)₂Pz]Ag}₃ (FIG. 1, [Ag—H]₃) reported previously,¹⁶ is a strong π-acid and display rich π-acid/π-base chemistry with unsaturated hydrocarbons leading to sandwich complexes of various types.¹⁷ It also serves as a sensor for arenes such as benzene and toluene.¹⁸⁻¹⁹ With o-terphenyl,²⁰ it produces a white light emitting material while the treatment of [Ag—H]₃ with phenylacetylene produces a Ag₁₃ cluster with the breakup of the Ag₃N₆ core.²¹ The silver complex has also been utilized in the desulfurization of fossil fuels.²²

In contrast to the aromatic hydrocarbons, the chemistry of industrially relevant gaseous hydrocarbons such as ethylene with silver pyrazolates has not been explored. Silver-ethylene complexes are of particular interest since silver is the metal of choice for partial oxidation of ethylene, which is a major industrial process.^{23, 24} They are challenging to stabilize and quite labile due to the relatively weak silver(I)-ethylene interactions.²⁵⁻³¹ Reversible binding of ethylene to silver, however, is valuable in applications such as the separation of ethylene from ethylene-ethane mixtures using silver complexes and silver-doped materials.³²⁻³⁴ The copper(I) analogs of [Ag—H]₃ such as {[4-R-3,5-(CF₃)₂Pz]Cu}₃ ([Cu—R]₃, R=H, Br, CF₃) are effective in the selective separation of ethylene from ethane containing mixtures.^{6, 7}

Motivated by the fundamental interest and novelty, an in-depth study was conducted of ethylene chemistry of silver(I) pyrazolates {[4-R-3,5-(CF₃)₂Pz]Ag}₃ ([Ag—R]₃, R=H, Br, CF₃) with different pyrazolyl ring substituents that also utilizes solid-gas³⁵⁻³⁸ synthesis and in situ powder X-ray diffraction (PXRD) measurements at 17-BM beamline at the Argonne National Laboratory (ANL) advanced photon source. As evident from the following account, this undertaking was successful and led to the stabilization of an unusual trinuclear silver-ethylene complex in a crystalline state. Additionally uncovered were two unprecedented dinuclear silver-ethylene complexes with bridging pyrazolates, of which, only one could be obtained via a traditional solution method.

### Results and Discussion

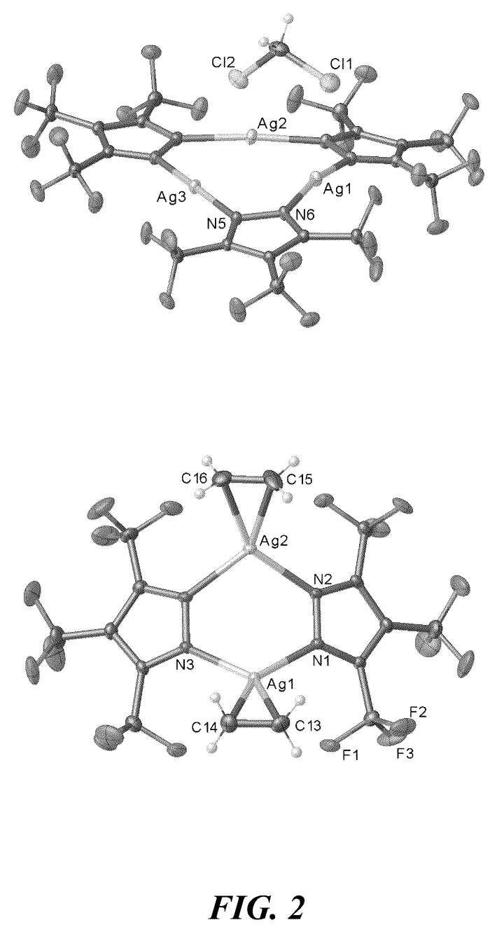

Traditional solution chemistry. The per-fluorinated silver(I) complex {[3,4,5-(CF₃)₃Pz]Ag}₃ ([Ag—CF₃]₃) was utilized first for this purpose because it possesses powerful Lewis acidic silver sites and is expected to be more reactive towards ethylene compared to the less-fluorinated analogs. The [Ag—CF₃]₃ was obtained very conveniently via a reaction between the corresponding pyrazole [3,4,5-(CF₃)₃Pz]H³⁹ and silver(I) oxide. It is a colorless, air-stable solid and has been characterized by several techniques including NMR spectroscopy, and single crystal and powder X-ray crystallography. It crystallizes with a molecule of dichloromethane in the asymmetric unit (FIG. 2) and displays short intermolecular Ag⋅⋅⋅Cl and Ag⋅⋅⋅F contacts. There are no argentophilic interactions as observed in [Ag—H]₃ or electron-rich systems like {[3,5-(Ph)₂Pz]Ag}₃ and {[3,5-(i-Pr)₂Pz]Ag}₃.⁴⁰⁻⁴²

More importantly, [Ag—CF₃]₃ reacts with ethylene in CH₂Cl₂ at low temperatures and produces a product which can be crystallized from the same mixture at −25° C. under an ethylene blanket (Scheme 1). The variable temperature ¹⁹F NMR spectroscopic data show that this transformation takes place below −10° C. in CD₂Cl₂. The analysis of crystalline solid using single crystal X-ray diffraction reveals that it is a dinuclear species [Ag-CF₃·(C₂H₄)]₂ (FIG. 2), and a rare isolable silver-ethylene complex.^{27, 29, 30, 43-59} Solid samples, however, lose ethylene rapidly upon removal from the ethylene atmosphere at room temperature and return to the ethylene-free trimer form [Ag—CF₃]₃ (Scheme 1).

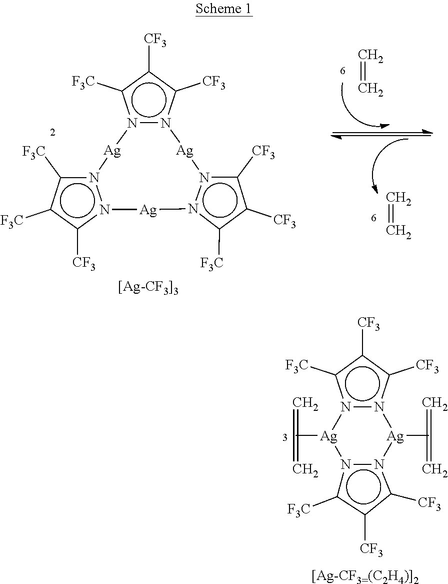

There are two chemically similar but crystallographically different molecules of [Ag-CF₃·(C₂H₄)]₂ in the asymmetric unit. The silver sites are trigonal planar and Ag₂N₄ cores adopt a boat shape. Although there are no analogous dinuclear silver-ethylene complexes for a direct comparison, a few silver-ethylene complexes such as [PhB(3-(CF₃)Pz)₃]Ag(C₂H₄)⁴⁶ and {[H₂C(3,5-(CF₃)₂Pz)₂]Ag(C₂H₄)}[SbF₆]²⁷ with a three coordinate silver sites supported by N-donor ligands are known. The average Ag—N(2.231 Å) and Ag—C(2.282 Å) distances of [Ag-CF₃·(C₂H₄)]₂ are similar to those observed in [PhB(3-(CF₃)Pz)₃]Ag(C₂H₄) (av. Ag—N and Ag—C are 2.261 and 2.264 Å, respectively).

Next, the related {[3,5-(CF₃)₂Pz]Ag}₃ ([Ag—H]₃)¹⁶ was studied, which is a molecule based on less fluorinated pyrazolate possessing relatively less electrophilic silver sites. The attempts to observe the silver-ethylene complex from a reaction between [Ag—H]₃ and ethylene in CH₂Cl₂ solution were unsuccessful even at −50° C. It is understandable since ethylene-silver bonds in general are quite weak while the Ag—N bonds in [Ag—H]₃ are relatively strong considering that it features a better electron-donating pyrazolate⁶⁰ than the one present in [Ag—CF₃]₃.

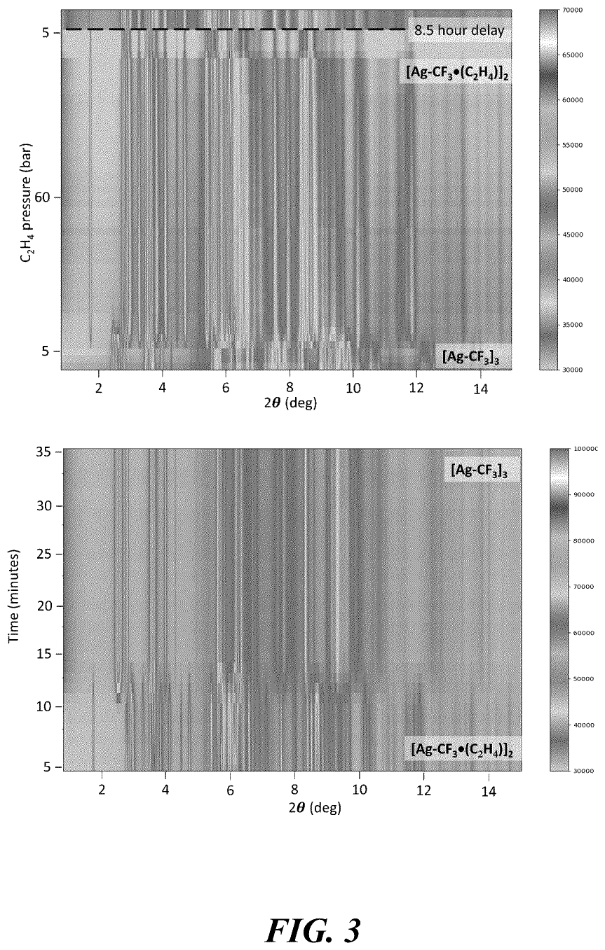

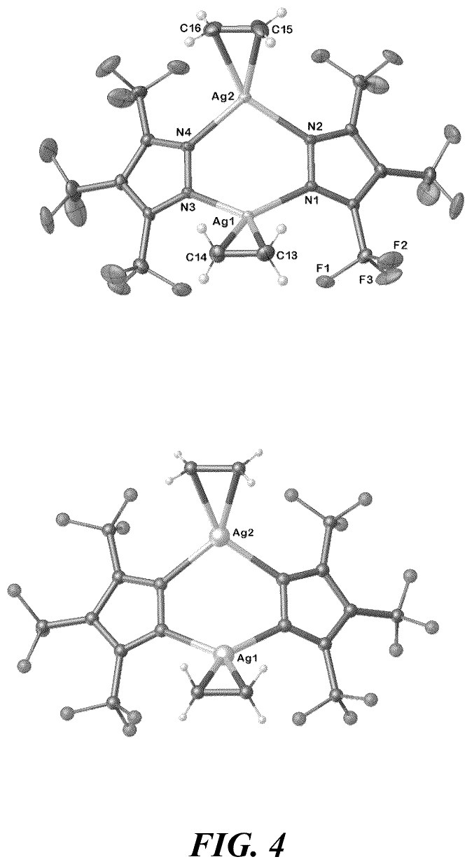

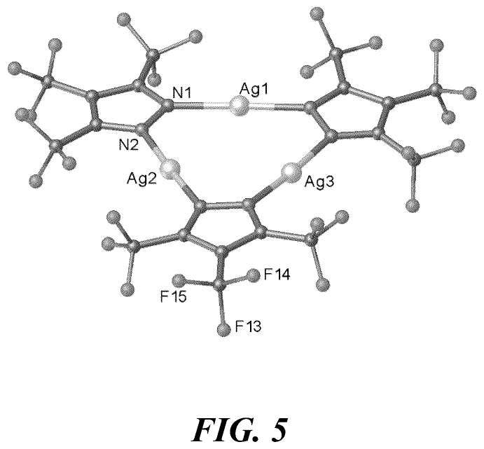

In situ solid-gas chemistry: Next, these processes were investigated, using solid materials and study the progress of the reaction “live” using in situ PXRD at ANL synchrotron beamline. Recent developments show that in situ and solid-gas chemistry are valuable techniques that enable synthesis and characterization of organometallic species that are difficult or impossible to observe under solution-phase conditions.³⁵⁻³⁸ Remarkably, crystals of [Ag—CF₃]₃ upon exposure to ethylene (3-5 bar at 295K, FIG. 3), converted smoothly to the same dinuclear silver-ethylene complex [Ag-CF₃·(C₂H₄)]₂ (FIG. 4), mimicking process that occurs in solution. The PXRD based molecular structure of the solid-gas generated [Ag-CF₃·(C₂H₄)]₂ is very similar to that obtained from traditional solution chemistry (and single crystal X-ray crystallography, FIG. 2). It is a reversible process (as in the solution) and affords ethylene free precursor [Ag—CF₃]₃ (FIG. 5) upon purging crystalline [Ag-CF₃·(C₂H₄)]₂ with helium at 295K (FIG. 3). Furthermore, these solid-gas reactions, despite the complexity and break-up and formation of several bonds and rearrangement of molecular fragments, are quite fast as evident from the PXRD patterns. Although the progress of both the forward and reverse reaction involving [Ag—CF₃]₃ can be followed using in situ PXRD, the trimer-dimer transition under the conditions noted above generates the products directly with no evidence of crystalline phases attributable to intermediates.

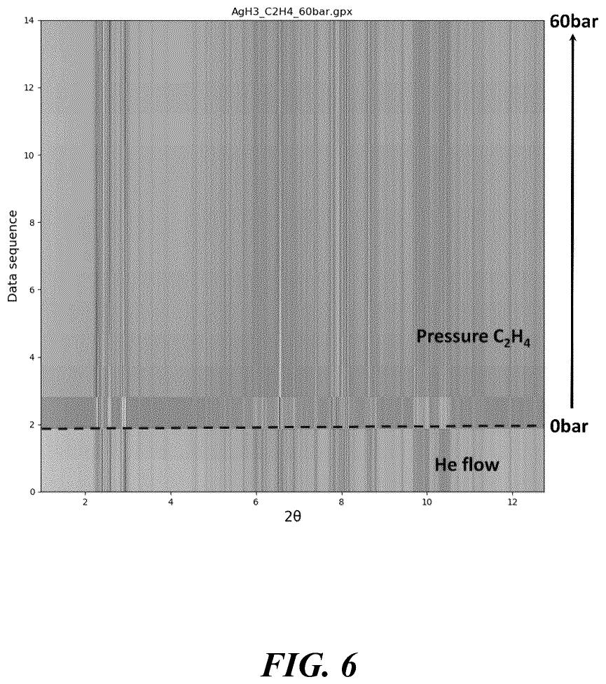

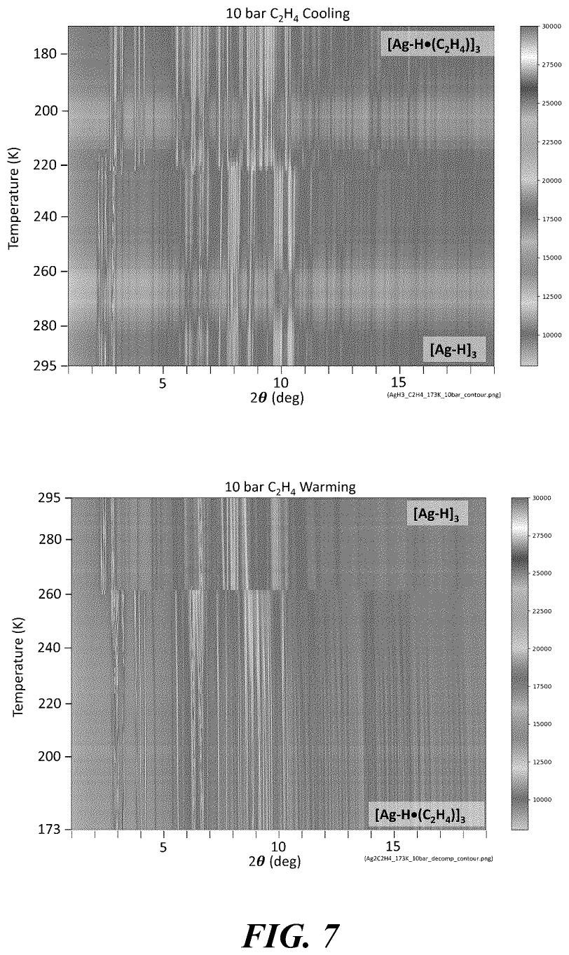

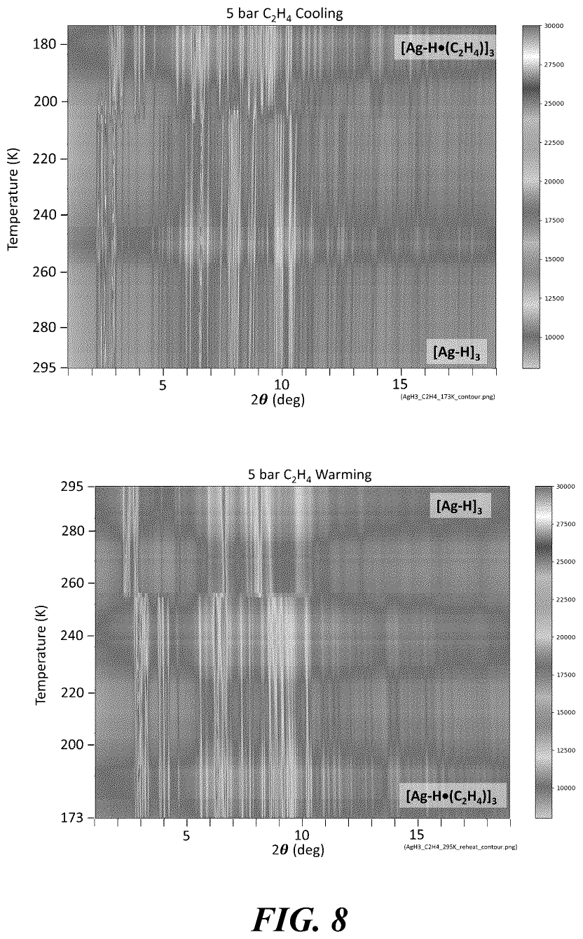

To see if transient species can be detected, in situ studies of the less reactive [Ag—H]₃ with ethylene were conducted. In contrast to [Ag—CF₃]₃, the reaction of solid [Ag—H]₃ with ethylene did not proceed at 295K even under high ethylene pressure up to 60 bar (FIG. 6), nor when cooled to 173K under ˜1 bar of ethylene flow. However, the solid-gas reaction proceeded as the temperature of polycrystalline [Ag—H]₃ was lowered while subjecting the sample to higher ethylene pressure. Specifically, the transformation was evident from the in situ PXRD experiment as the PXRD lines of [Ag—H]₃ started to disappear around 223K at 10 bar (or 206K at 5 bar) of ethylene with the generation of a new crystalline phase (FIGS. 7 and 8). This new phase did not change even upon further cooling to 173K under ethylene. The process of ethylene uptake by [Ag—H]₃ was reversible, and the product converted back to ethylene free [Ag—H]₃ upon warming to about 262K even under 10 bar of ethylene (FIG. 7). The PXRD data analysis revealed the structure of the product (illustrated in Scheme 2), which turned out to be not the dinuclear species encountered with [Ag—CF₃]₃, but an unusual silver-ethylene complex {[3,5-(CF₃)₂Pz]Ag(C₂H₄)}₃ ([Ag-H·(C₂H₄)]₃) that retains the trinuclear form.

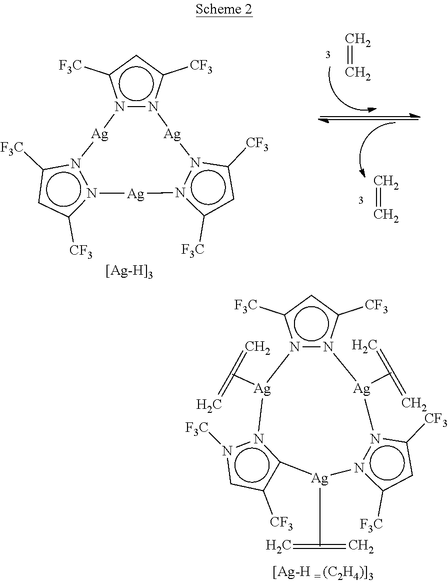

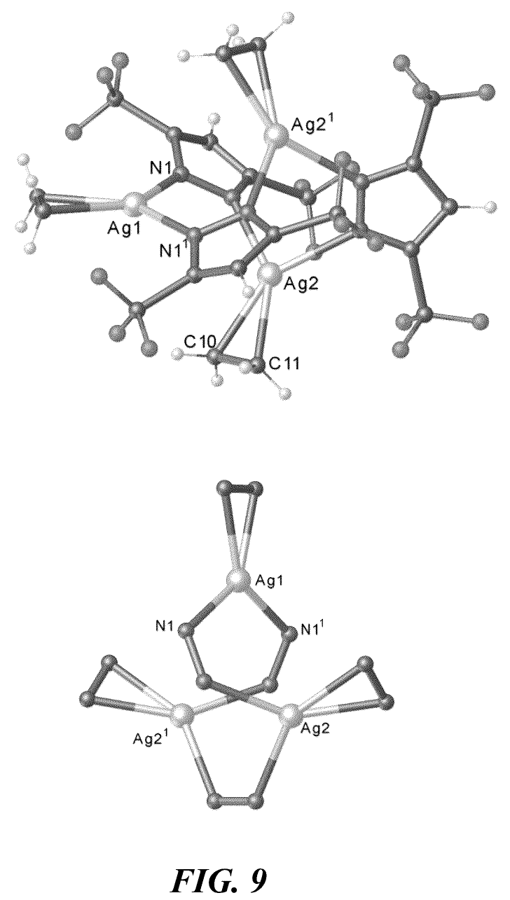

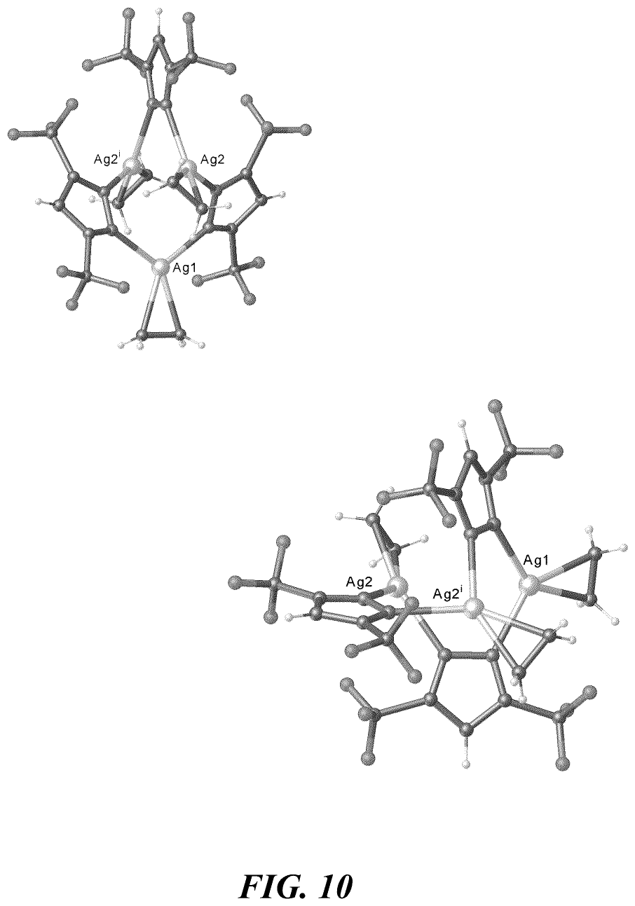

The molecular structure of this unprecedented species [Ag-H·(C₂H₄)]₃ is illustrated in FIG. 9 and FIG. 10. It is a trinuclear silver complex featuring a nine-membered Ag₃N₆ metallacycle, and three trigonal-planar silver-ethylene sites. The Ag₃N₆ core of [Ag-H·(C₂H₄)]₃ displays significant puckering compared to the planar configuration found in [Ag—H]₃ (and the related [Ag—CF₃]₃, see FIG. 5).¹⁶ This large deviation from planarity is a result of the interaction of ethylene with silver sites from opposite faces, but the interactions are perhaps not strong enough to break the Ag—N bonds at low-temperature conditions. The compound [Ag-H·(C₂H₄)]₃ may possibly be a model for a likely intermediate present in more facile reaction of [Ag—CF₃]₃ with ethylene, just prior to the breakup of trimers to produce the corresponding dinuclear metal-ethylene complexes.

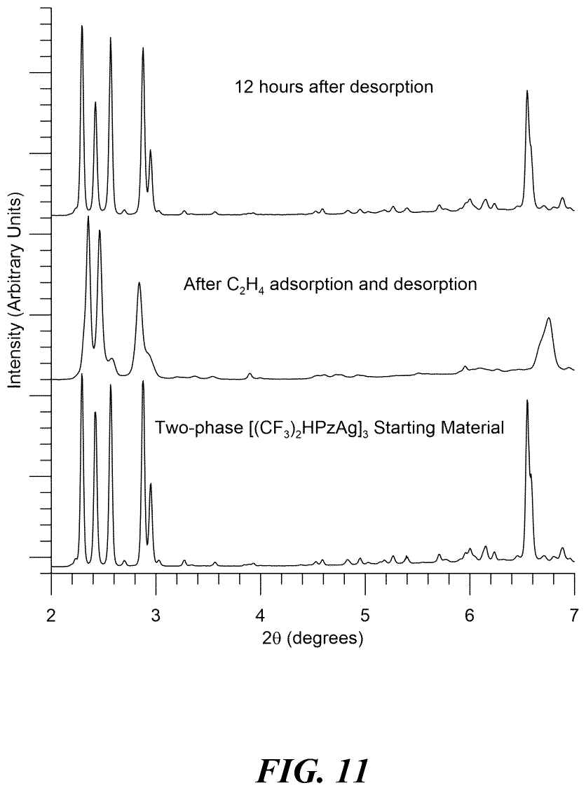

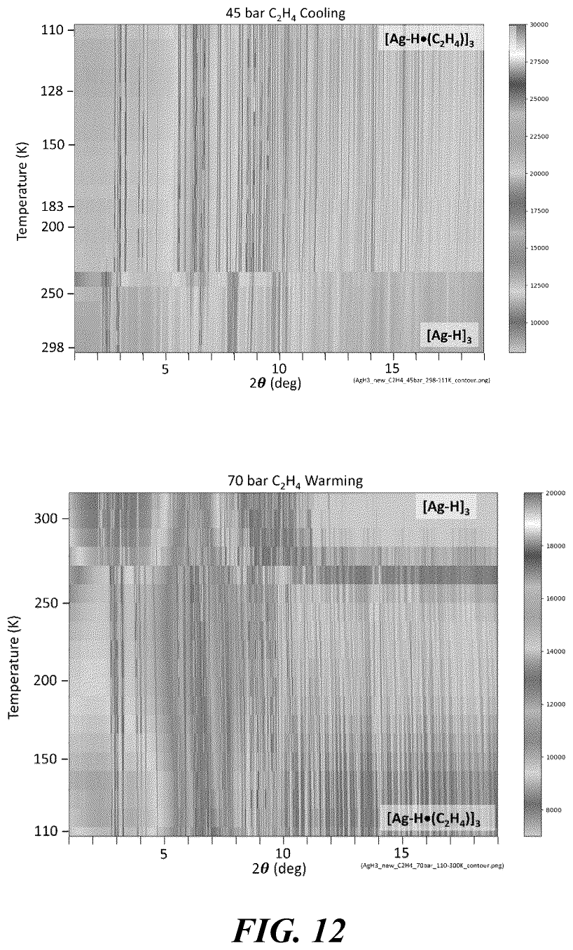

Postulating that this ethylene loaded trimer phase [Ag-H·(C₂H₄)]₃ might be a transition state between unloaded trimer and loaded dimer phases observed for other metal pyrazolates, experiments were carried out at even higher pressures and lower temperatures to see if a further transition to a loaded dimer “[Ag-H·(C₂H₄)]₂” could be observed. First, the in situ PXRD data were collected at 45 bar of C₂H₄ from room temperature down to 110 K (just above the freezing point of C₂H₄). The pressure was then increased to 70 bar of ethylene and the sample warmed to room temperature (which led to [Ag—H]₃ formation). No evidence of a new crystalline phase was observed under either conditions (see FIGS. 11 and 12).

Encouraged by the success with [Ag—H]₃ that led to the characterization of a rare species in the ethylene bound yet pre trimer--dimer transformation stage, the chemistry of [Ag—Br]₃ with ethylene was probed. Note that these planar, trinuclear metal adducts displayed different extended structures and therefore, the outcome of solid-state chemistry with ethylene was not necessarily predictable through extrapolation. For example, in contrast to [Ag—H]₃ which crystallizes forming zig-zag columns with argentophilic interactions,^{16, 61} [Ag—Br]₃ trimers formed extended structures with inter-trimer Ag⋅⋅⋅Br contacts⁶² (while [Ag—CF₃]₃ reported here shows inter-trimer Ag⋅⋅⋅F interactions between trimers).

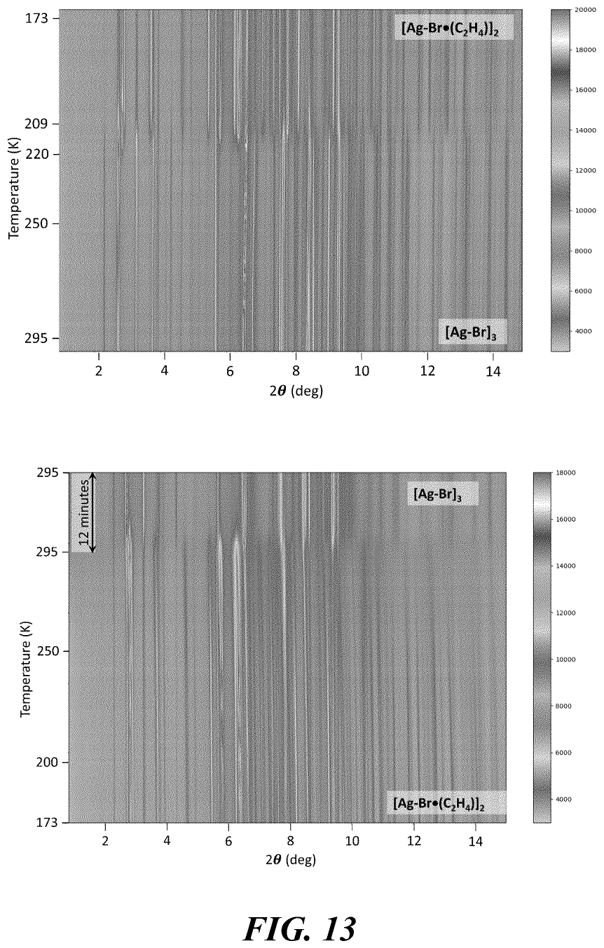

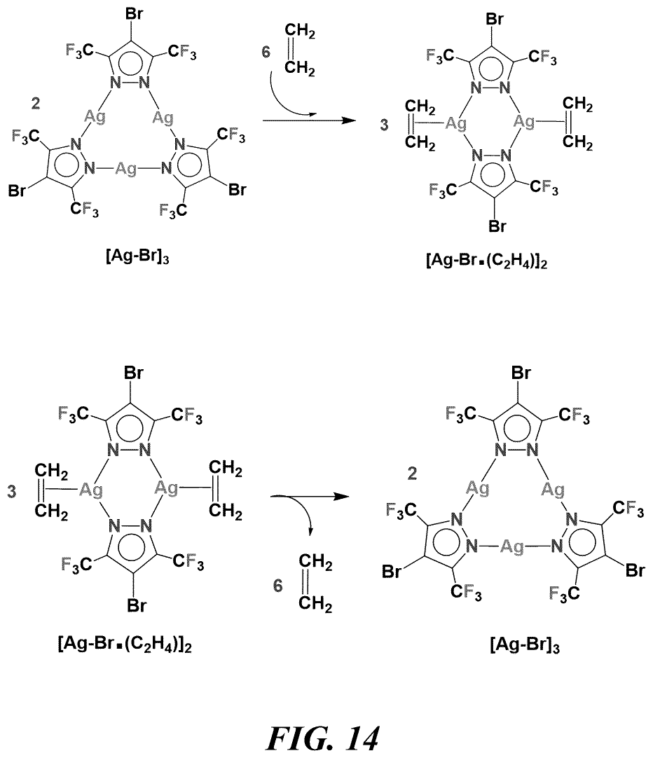

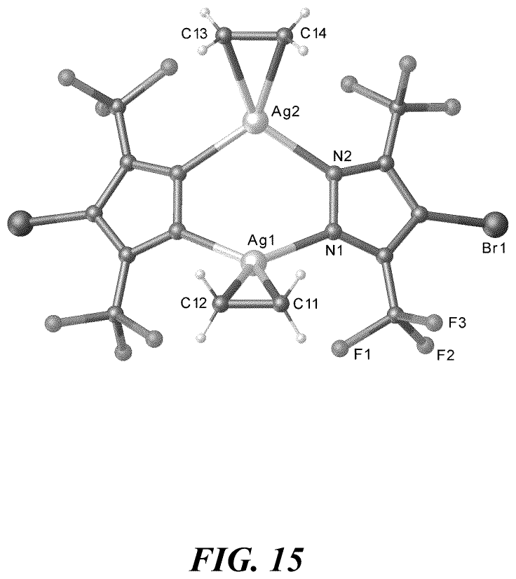

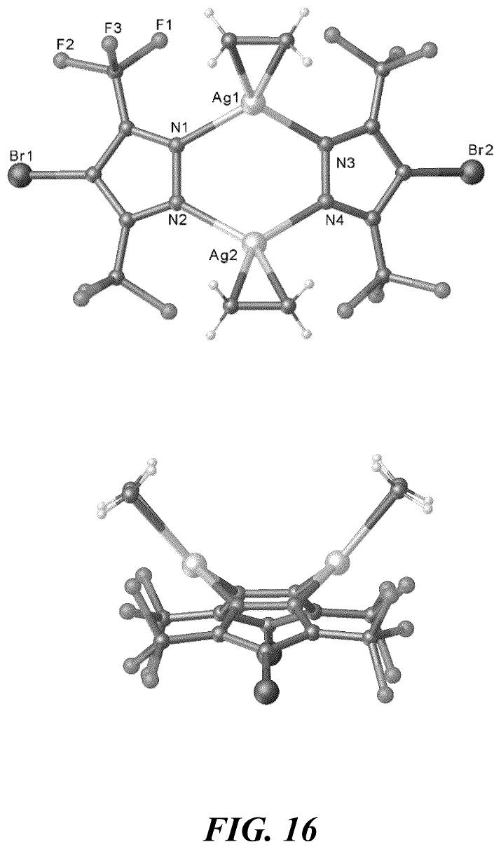

Traditional solution chemistry with ˜1 bar ethylene did not yield an isolable silver-ethylene complex from [Ag—Br]₃ in CH₂Cl₂. The in situ PXRD data of the solid-gas reaction of polycrystalline [Ag—Br]₃ also did not show any phase changes even at 173K under flow of ethylene (˜1 bar). However, at 10 bar of ethylene, a notable change was observed at 220K (FIGS. 13 and 14). Data analysis indicated that it directly progressed to the dimer stage producing {[4-Br-3,5-(CF₃)₂Pz]Ag(C₂H₄)}₂ ([Ag-Br·(C₂H₄)]₂ (FIGS. 15 and 16), which is in contrast to the [Ag—H]₃ chemistry but similar to that observed with [Ag—CF₃]₃ and ethylene. Upon warming, [Ag-Br·(C₂H₄)]₂ lost ethylene and returned to the precursor trimer at 295K, even under 10 bar of ethylene (FIGS. 13 and 14). The dinuclear silver(I) ethylene complex [Ag-Br·(C₂H₄)]₂ adopted a slightly deeper boat configuration with a closer Ag⋅⋅⋅Ag separation (3.35(2) A) within the six-membered Ag₂N₄ core relative to that observed with [Ag-CF₃·(C₂H₄)]₂ (which has Ag⋅⋅⋅Ag separations at 3.49(2) Å). Ethylene ligands are h²-bonded to silver sites, as expected. Overall, trinuclear [Ag—Br]₃ and [Ag—CF₃]₃ show unprecedented ethylene triggered solid-gas chemistry leading to dinuclear silver-ethylene complexes featuring Ag₂N₄ cores while [Ag—H]₃ enabled the observation of an ethylene bound silver trimer that retains the metalacyclic Ag₃N₆ core.

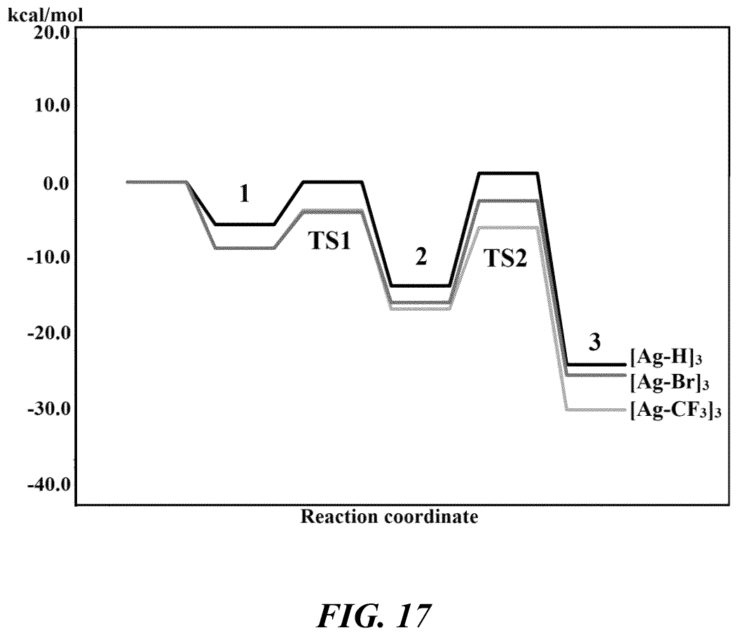

Computational study. In order to further understand ethylene driven molecular reorganization processes described above, a detailed computational study of ethylene reactions of [Ag—CF₃]₃, [Ag—Br]₃ and [Ag—H]₃ were conducted (see FIG. 17). The Gibbs free energy profiles at 298K were computed to uncover reaction paths at room temperature in the molecular calculations at the TZ2P/BP86-D3 level of theory. For this purpose, thermodynamic quantities from vibrational frequencies accounting for enthalpy and entropy changes for the proposed reaction mechanism were obtained. As the first step (FIG. 17, 1), the formation of an adduct between the trinuclear silver pyrazolate and three molecules of C₂H₄ was predicted, prior to the deformation of the Ag₃N₆ core as a transition state (TS1), which is further relaxed to the intermediate 2 (such as [Ag-H·(C₂H₄)]₃), The formation of 2 involves a computed Gibbs free energy (298.15 K) of −16.8, −15.9 and −13.7 kcal/mol, respectively, in comparison to the initial reactants, for [Ag—CF₃]₃, [Ag—Br]₃ and [Ag—H]₃. The observed deformation of the Ag₃N₆ core from precursors to the intermediates is not favored in the absence of ethylene, by about 50 kcal/mol (Table S7) for all the species, showing that such processes is driven exclusively by the initial coordination of C₂H₄ to the bare Ag₃N₆ core (step 1). The process of forming [Ag-R·(C₂H₄)]₃ from [Ag—R]₃ and gaseous ethylene (R=H, Br, CF₃) is not favorable entropically and can be influenced significantly by lower temperatures. Thus, intermediate 2 is more likely to be characterized, especially at lower temperature, as experimentally realized in this work in the reaction involving [Ag—H]₃.

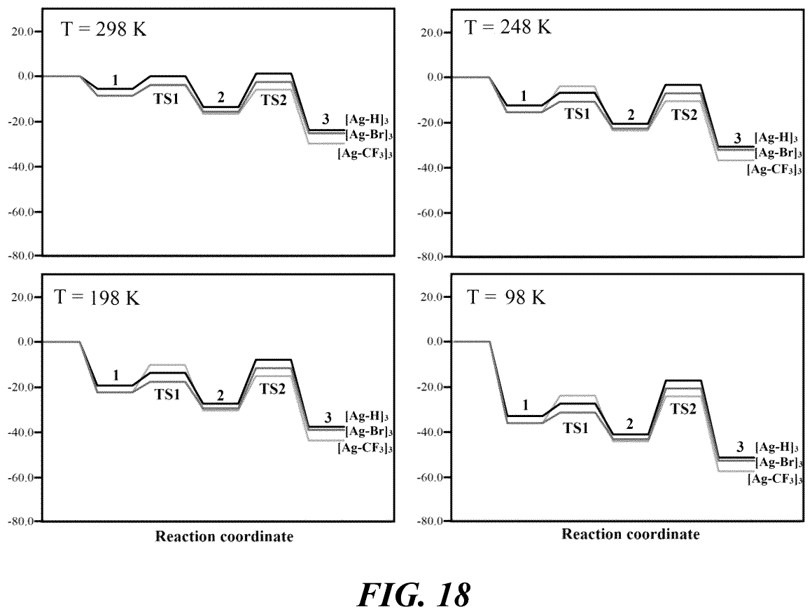

Intermediate 2 is a key step prior to the trimer→dimer transformation. After the formation and relaxation of this intermediate, the next step is to release one [Ag-R·(C₂H₄)]unit (i.e., ethylene bound metal-pyrazolate) given as the second transition state (TS2), which is the rate-determinant step leading to the dimer. Calculations of the bonding energy of Ag₂N₄—AgN₂ for —H, —Br and —CF₃, indicate that it is easier to break-up [Ag—CF₃]₃ and [Ag—Br]₃ species (−64.3 and −65.2 kcal/mol, respectively), in comparison to [Ag—H]₃ counterpart (−83.8 kcal/mol). From the Gibbs free energy profiles (FIG. 18), the activation barriers related to the 1→TS1 process can be evaluated, which amount to 5.0, 4.7, and 5.6 kcal/mol for —CF₃, —Br, and —H at 298 K, respectively. For the 2→TS2 process, the related values are 10.8, 13.4, and 14.9 kcal/mol, denoting a slightly larger activation barrier for the [Ag—H]₃ complex.

In the final step, the loss of a [Ag-R·(C₂H₄)]unit from [Ag-R·(C₂H₄)]₃, leads to the formation of one dimer species [Ag-R·(C₂H₄)]₂ (TS2), where the released unit further aggregates with another [Ag-R·(C₂H₄)]fragment from a parallel reaction, resulting in the formation of a second dimer species (3). Calculated Gibbs free energy for step 3 amounts to −30.1, −25.5, and −24.1 kcal/mol for [Ag—CF₃]₃, [Ag—Br]₃ and [Ag—H]₃, respectively. Overall, [Ag-H·(C₂H₄)]₂ formation is slightly less energetically favorable process, while [Ag-CF₃·(C₂H₄)]₂ formation is the most facile, which is consistent with the experimental observations, and denoted by the slightly less stabilized transition states and activation barriers, in addition to the bonding energy of Ag₂N₄—AgN₂ fragments prior formation of TS2. The formation of trinuclear-tris-ethylene intermediate 2 is favored at lower temperatures but the experimental conditions must be just right to trap this species before it breaks-up to even more energetically favorable dimers 3. The silver(I) and [3,5-(CF₃)₂Pz]⁻ ligand combination provides the ideal ingredients to trap the elusive species [Ag-H·(C₂H₄)]₃.

In summary, after a careful investigation that involved strategic variations of pyrazolyl ring substituents and solid-gas synthesis under different temperature-pressure combinations, and synchrotron based, in situ PXRD, the trinuclear silver-ethylene complex {[3,5-(CF₃)₂Pz]Ag(C₂H₄)}₃ ([Ag-H·(C₂H₄)]₃) was trapped and structurally characterized, showing a severely distorted, yet intact Ag₃N₆ core, that can be viewed as a model for fleeting intermediates likely exist in ethylene driven, trimer-dimer transformations observed in related [Ag—CF₃]₃ and [Ag—Br]₃ systems. Furthermore, this study reveals for the first time, ethylene triggered structural transformations of trinuclear silver(I) pyrazolates in the solid-state leading to dinuclear species {[3,4,5-(CF₃)₃Pz]Ag(C₂H₄)}₂ ([Ag-CF₃·(C₂H₄)]₂) and {[4-Br-3,5-(CF₃)₂Pz]Ag(C₂H₄)}₂ ([Ag-Br·(C₂H₄)]₂), and molecular structures of two rare dinuclear, silver-ethylene complexes. This investigation also demonstrates the power of in situ synthesis over traditional solution chemistry for the isolation of labile species. Computational studies indicated that the silver(I) and [3,5-(CF₃)₂Pz]⁻ ligand combination provides the ideal ingredients to stabilize [Ag-H·(C₂H₄)]₃.

### Materials and Methods

General Information. All manipulations were carried out under an atmosphere of purified nitrogen using standard Schlenk techniques or in a MBraun glovebox equipped with a −25° C. refrigerator. Solvents were purchased from commercial sources and purified before use. NMR spectra were recorded at 25° C. on a JEOL Eclipse 500 spectrometer (¹H, 500.16 MHz ¹³C, 125.78 MHz, and ¹⁹F, 470.62 MHz) unless otherwise noted. ¹H and ¹³C NMR spectra are referenced to the solvent peak (¹H; CDCl₃ δ 7.26, ¹³C; CDCl₃ δ 77.16). ¹H NMR coupling constants (J) are reported in Hertz (Hz) and multiplicities are indicated as follows: s (singlet), d (doublet), t (triplet), m (multiplet). ¹⁹F NMR values were referenced to external CFCl₃. Melting points were obtained on a Mel-Temp II apparatus and were not corrected. Elemental analyses were performed using a Perkin-Elmer Model 2400 CHN analyzer. IR spectra were collected at room temperature on a Shimadzu IR Prestige-21 FTIR containing an ATR attachment using pure liquid or solid materials, with instrument resolution at 2 cm⁻¹. Raman data were collected on a Horiba Jobin Yvon LabRAM Aramin Raman spectrometer with a HeNe laser source of 633 nm, by placing pure solid materials on a glass slide. All other reactants and reagents were purchased from commercial sources. Heating was accomplished by either a heating mantle or a silicone oil bath. The 3,4,5-(CF₃)₃PzH was prepared via reported methods.

Synthesis of {[3,4,5-(CF₃)₃Pz]Ag}₃ ([Ag—CF₃]₃). Freshly dried 3,4,5-(CF₃)₃PzH (300 mg, 1.10 mmol) and Ag₂O (211 mg, 0.91 mmol) were placed in a Schlenk flask attached to a reflux condenser, slowly heated to 125-130° C. and kept for 4 hours while stirring, or until unreacted pyrazole stops condensing on the walls the flask. A heat gun was used to meltdown the pyrazole condensed on the wall of the Schlenk flask, as needed. Excess 3,4,5-(CF₃)₃PzH was sublimated off, the product was extracted into dichloromethane and filtered through a bed of Celite. The filtrate was collected, and solvent was removed under reduced pressure to obtain {[3,4,5-(CF₃)₃Pz]Ag}₃ ([Ag—CF₃]₃) as a white powder. X-ray quality crystals were grown from dichloromethane at −20° C. Yield: 91%. M.p.: 260° C. Anal. Calc. for C₁₈N₆F₂₇Ag₃: C, 19.02; H, 0.00; N, 7.39. Found: C, 18.98; H, <0.1; N, 7.41. ¹⁹F NMR (in CDCl₃): δ (ppm) −54.94 (br s), −60.63 (q, 7.2 Hz). ¹³C{¹H}NMR (in CDCl₃): δ (ppm) 110.6 (q, ²J(C,F)=42.0 Hz, CCF₃), 119.2 (q, ¹J(C,F)=270.3 Hz, CF₃), 120.7 (q, ¹J(C,F)=262.7 Hz, CF₃), 143.5 (q, ²J(C,F)=36.0 Hz, CCF₃). Raman (cm⁻¹): 3148, 1594, 1536, 1502, 1453, 1349, 1154, 1088, 1000, 985, 845.

Synthesis of {[3,4,5-(CF₃)₃Pz]Ag(C₂H₄)}₂. {[3,4,5-(CF₃)₃Pz]Ag}3 (200 mg, 0.18 mmol) was dissolved in 10-12 mL of CH₂Cl₂, ethylene was bubbled for approximately 10 minutes. The ethylene saturated solution was kept at −25° C. to obtain X-ray quality colorless crystals of {[3,4,5-(CF₃)₃Pz]Ag(C₂H₄)}₂ ([Ag-CF₃·(C₂H₄)]₂). Yield: 96%. ¹⁹F NMR (in CDCl₃ at −50° C.): δ (ppm) −60.00 (3,5-CF₃) and −54.97 (4-CF₃). ¹H NMR (in CDCl₃ at −50° C.): δ (ppm) 5.42.

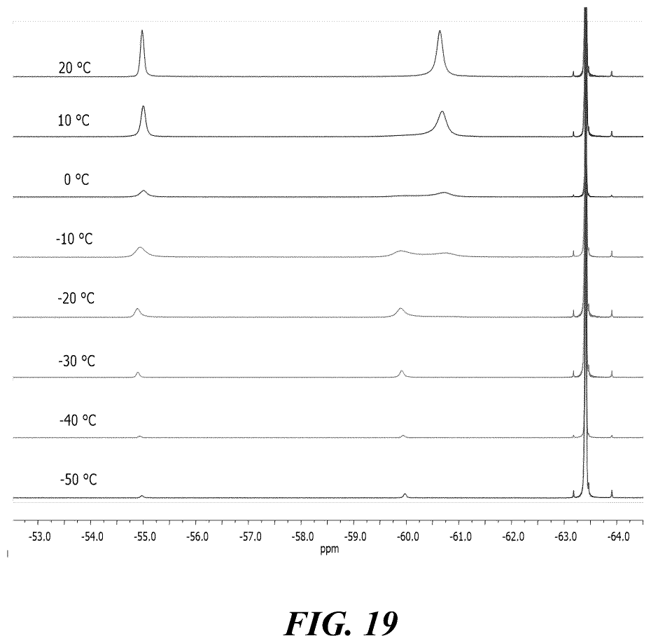

NMR Studies of solution-state equilibria for the [Ag-CF₃·(C₂H₄)]₂ reaction with ethylene. As evident from these spectra, at 20° C. in CDCl₃, [Ag-CF₃·(C₂H₄)]₂ completely dissociates into [Ag—CF₃]₃ and free ethylene. Therefore, ¹⁹F NMR peaks corresponding to [Ag—CF₃]₃ species can be observed at 20° C. (−60.64 and −54.98 for 3,5-CF₃ and 4-CF₃, respectively). Results are shown in FIG. 19.

From 0° C. to −20° C. signals for both [Ag-CF₃·(C₂H₄)]₂ and [Ag—CF₃]₃ was observed in various ratios.

From −30° C. to −50° C. only signals corresponding to [Ag-CF₃·(C₂H₄)]₂ was observed (−60.00 and −54.97 ppm corresponding to 3,5-CF₃ and 4-CF₃ groups, respectively).

No clear shift was observable, likely due to highly fluxional Ag-ethylene interaction although the ¹⁹F NMR data indicated the formation of [Ag-CF₃·(C₂H₄)]₂ from [Ag—CF₃]₃/ethylene when the temperature is lower than −10° C.

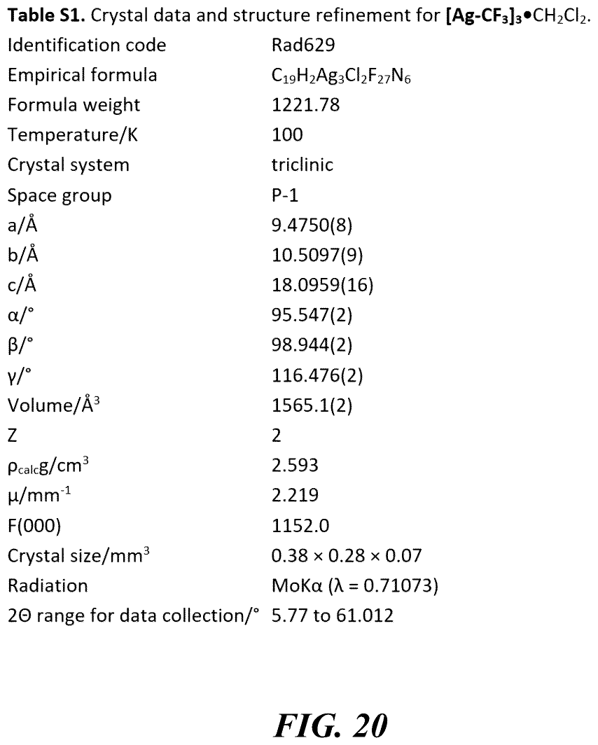

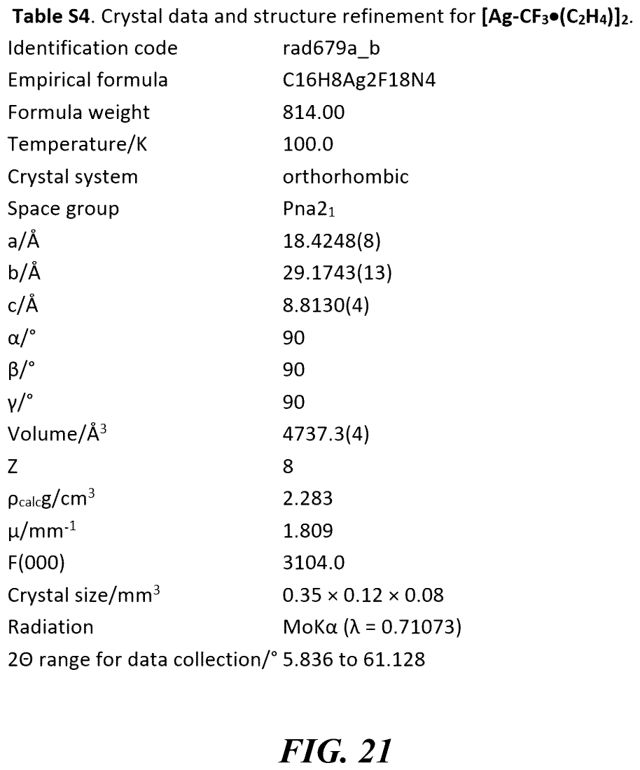

Single crystal X-ray crystallography. A suitable crystal covered with a layer of hydrocarbon/Paratone-N oil was selected and mounted on a Cryo-loop, and immediately placed in the low-temperature nitrogen stream. The X-ray intensity data were measured at 100(2) K on a Bruker D8 Quest with a Photon 100 CMOS detector equipped with an Oxford Cryosystems 700 series cooler, a graphite monochromator, and a Mo Kα fine-focus sealed tube (λ=0.71073 Å). Intensity data were processed using the Bruker Apex program suite. Absorption corrections were applied by using SADABS.⁶³ Initial atomic positions were located by SHELXT,⁶⁴ and the structures of the compounds were refined by the least-squares method using SHELXL⁶⁵ within Olex2 GUI.⁶⁶ All the non-hydrogen atoms were refined anisotropically. There are two molecules of [Ag-CF₃·(C₂H₄)]₂ in the asymmetric unit. It was solved and refined in Pna2₁ space group as an inversion twin. The Pccn space group for [Ag-CF₃·(C₂H₄)]₂ was tested, as suggested by the CheckCif but the refinement indicators were significantly poor. The hydrogen atoms of the ethylene groups of [Ag-CF₃·(C₂H₄)]₂ was located using difference Fourier maps, included, and refined freely with isotropic displacement parameters. The hydrogen atoms of [Ag—CF₃]₃·CH₂Cl₂ were included in their calculated positions and refined as riding on the atoms to which they are joined. X-ray structural figures were generated using Olex2.⁶⁶ Results of crystal data and structure refinement for [Ag—CF₃]₃·CH₂Cl₂ and [Ag-CF₃·(C₂H₄)]₂ are shown in FIGS. 20 and 21, respectively.

Single crystal X-ray crystallography of [Ag—CF₃]₃·CH₂Cl₂. The analysis of the crystal of [Ag—CF₃]₃ obtained from dichloromethane by single crystal X-ray diffraction show that it crystallizes as [Ag—CF₃]₃·CH₂Cl₂ from dichloromethane in P1 space group with chlorine atoms facing silver sites, with Ag—Cl contacts shorter than the van der Waals contact separation of Ag and Cl (3.47 Å). This suggested that the silver sites are quite Lewis acidic even to interact with weakly donating CH₂Cl₂ which is not surprising considering the number of electron-withdrawing fluorinated substituents on the ligand backbone. The solvent molecules in the crystal lattice, however, can be removed easily under reduced pressure to obtain [Ag—CF₃]₃, of which structure was confirmed by analyzing the material using powder X-ray diffraction.

In situ synchrotron powder diffraction data collection (PXRD). In situ powder x-ray diffraction data (PXRD) from solid samples in ethylene or helium were collected using the monochromatic X-rays available at the Advanced Photon Source, Argonne National Laboratory, beamline 17-BM. A circular incident beam (300 m diameter) was used a width of approximately 0.45 Å wavelength for experiments in conjunction with a VAREX 4343 amorphous-Si flat panel detector. Samples of were loaded into 1.0 mm quartz capillaries with glass wool on either side. The capillary with sample was then loaded into a gas flow cell,⁶⁷ to perform in situ PXRD experiments. At one end the gas cell was connected to a two-way valve which allowed changing between a 1 atm helium flow and a high-pressure syringe pump (Teledyne ISCO 500D) which was filled with ethene gas. Pressure was monitored at the other end of the gas flow cell to make sure that there was no blockage or leak during the measurements. Pressures quoted are absolute, so a purge of helium or ethylene is a pressure of 1 bar. Prior to measurements, all samples were activated with supercritical ethylene (295 K, 55 bar) as described.⁶

### Data Processing.

The raw images were processed within GSAS-II,⁶⁸ refining the sample-to-detector distance and tilt of the detector relative to the beam based on data obtained for a LaB₆ standard.⁶⁹ Collected 2D detector images from in situ powder diffraction data sets were reduced to 1D datasets of intensity vs. diffraction angle 2□ with GSAS-II. Plots showing the evolution of the PXRD data were prepared with the same software, in which the diffracted intensity is represented by a color scale. In call cases, time runs from the bottom to the top of the image. Individual scans were analyzed using Topas-Academic software.⁷¹ Structures from powder data were determined by simulated annealing, using the single crystal structures as a starting point. The pyrazole groups were incorporated as rigid bodies based on the average geometries from analogous single crystal data, with the CF₃ rotations freely refined. Selected bond distances, angles, and torsions were refined as appropriate. The unexpected structure of [Ag-H·(C₂H₄)]₃ was initially hypothesized from the volume of the indexed unit cell and was solved by repeated cycles of model building and refinement.

CIF files (PXRD based and from in situ synthesis) of previously unknown dinuclear [Ag-Br·(C₂H₄)]₂ and trinuclear [Ag-H·(C₂H₄)]₃ silver-ethylene complexes, along with the trinuclear precursor [Ag—CF₃]₃ for which a single crystal was not available (for the latter, single crystal data have CH₂Cl₂ as solvents of crystallization), have been deposited at the Cambridge Crystallographic Data Centre with CCDC numbers 2267047, 2266818, and 2266816 respectively.

### Computational Studies and Results

All calculations were carried out by using relativistic DFT methods employing the ADF code⁷¹ with the all-electron triple-ζ Slater basis set plus the double-polarization (STO-TZ2P) basis set in conjunction with the Becke-Perdew (BP86) functional⁷² within the generalized gradient approximation (GGA). London dispersion corrections were taken into account via the pair-wise Grimme3 approach.⁷³ Such level of theory is denoted as TZ2P/BP86-D3. Geometry optimizations were performed without any symmetry restrain, via the analytical energy gradient method implemented by Versluis and Ziegler,⁷⁴ with energy convergence criteria set at 10-4 Hartree, gradient convergence criteria at 10-4 Hartree/A and radial convergence of 10-3 Å. Thermodynamic properties were obtained from vibrational analysis. Scalar relativistic effects were considered through the ZORA Hamiltonian.⁷⁴

The interaction energy is further dissected into several chemically meaningful terms according to the Energy Decomposition Analysis (EDA) of Ziegler and Rauk,⁷⁵

$$Δ {E}_{int} = Δ {E}_{Pauli} + Δ {E}_{elstat} + Δ {E}_{orb} + Δ {E}_{disp}$$

Where the ΔE_{Pauli} term involves the electron repulsion between occupied orbitals from the different fragments. ΔEₑₗₛₜₐₜ and ΔE_{orb} are related to the stabilizing electrostatic and covalent character of the interaction, respectively. The contribution from dispersion interaction (ΔE_{disp}) is evaluated using the pairwise correction of Grimme (D3).

### REFERENCES

ADDIN EN.REFLIST 1. D. S. Sholl and R. P. Lively, Nature, 2016, 532, 435-437.
2. D. J. Safarik and R. B. Eldridge, Ind. Eng. Chem. Res., 1998, 37, 2571-2581.
3. P. Zhang, L. Yang, X. Liu, J. Wang, X. Suo, L. Chen, X. Cui and H. Xing, Nat. Commun., 2022, 13, 4928.
4. Y. Yang, L. Li, R.-B. Lin, Y. Ye, Z. Yao, L. Yang, F. Xiang, S. Chen, Z. Zhang, S. Xiang and B. Chen, Nat. Chem., 2021, 13, 933-939.
5. S. Du, J. Huang, M. R. Ryder, L. L. Daemen, C. Yang, H. Zhang, P. Yin, Y. Lai, J. Xiao, S. Dai and B. Chen, Nat. Commun., 2023, 14, 1197.
6. D. Parasar, A. H. Elashkar, A. A. Yakovenko, N. B. Jayaratna, B. L. Edwards, S. G. Telfer, H. V. R. Dias and M. G. Cowan, Angew. Chem., Int. Ed., 2020, 59, 21001-21006.
7. N. B. Jayaratna, M. G. Cowan, D. Parasar, H. H. Funke, J. Reibenspies, P. K. Mykhailiuk, O. Artamonov, R. D. Noble and H. V. R. Dias, Angew. Chem., Int. Ed., 2018, 57, 16442-16446.
8. J. Zheng, Z. Lu, K. Wu, G.-H. Ning and D. Li, Chem. Rev., 2020, 120, 9675-9742.
9. J. Luo, X. Luo, M. Xie, H.-Z. Li, H. Duan, H.-G. Zhou, R.-J. Wei, G.-H. Ning and D. Li, Nat. Commun., 2022, 13, 7771.
10. R. Galassi, M. A. Rawashdeh-Omary, H. V. R. Dias and M. A. Omary, Comments Inorg. Chem., 2019, 39, 287-348.
11. J.-P. Zhang, Y.-B. Zhang, J.-B. Lin and X.-M. Chen, Chem. Rev., 2012, 112, 1001-1033.
12. J. Zheng, H. Yang, M. Xie and D. Li, Chem. Commun., 2019, 55, 7134-7146.
13. M. A. Omary, M. A. Rawashdeh-Omary, M. W. A. Gonser, O. Elbjeirami, T. Grimes, T. R. Cundari, H. V. K. Diyabalanage, C. S. P. Gamage and H. V. R. Dias, Inorg. Chem., 2005, 44, 8200-8210.
14. A. A. Titov, O. A. Filippov, L. M. Epstein, N. V. Belkova and E. S. Shubina, Inorg. Chim. Acta, 2018, 470, 22-35.
15. R. Hahn, F. Bohle, W. Fang, A. Walther, S. Grimme and B. Esser, J. Am. Chem. Soc., 2018, 140, 17932-17944.
16. H. V. R. Dias, S. A. Polach and Z. Wang, J. Fluor. Chem., 2000, 103, 163-169.
17. H. V. R. Dias and C. S. Palehepitiya Gamage, Angew. Chem. Int. Ed., 2007, 46, 2192-2194.
18. M. A. Rawashdeh-Omary, M. D. Rashdan, S. Dharanipathi, O. Elbjeirami, P. Ramesh and H. V. R. Dias, Chem. Commun., 2011, 47, 1160-1162.
19. M. A. Omary, O. Elbjeirami, C. S. P. Gamage, K. M. Sherman and H. V. R. Dias, Inorg. Chem., 2009, 48, 1784-1786.
20. S.-Z. Zhan, F. Ding, X.-W. Liu, G.-H. Zhang, J. Zheng and D. Li, Inorg. Chem., 2019, 58, 12516-12520.
21. S.-K. Peng, Z. Lu, M. Xie, Y.-L. Huang, D. Luo, J.-N. Wang, X.-W. Zhu, X. Li, X.-P. Zhou and D. Li, Chem. Commun., 2020, 56, 4789-4792.
22. R. Liu, W. Zhang, D. Wei, J.-H. Chen, S. W. Ng and G. Yang, Dalton Trans., 2019, 48, 16162-16166.
23. H. Kestenbaum, A. Lange de Oliveira, W. Schmidt, F. Schiith, W. Ehrfeld, K. Gebauer, H. Löwe, T. Richter, D. Lebiedz, I. Untiedt and H. Züchner, Ind. Eng. Chem. Res., 2002, 41, 710-719.
24. J.-X. Liu, S. Lu, S.-B. Ann and S. Linic, ACS Catal., 2023, 13, 8955-8962.
25. R. H. Hertwig, W. Koch, D. Schröder, H. Schwarz, J. Hrušák and P. Schwerdtfeger, J. Phys. Chem., 1996, 100, 12253-12260.
26. H. V. R. Dias and C. J. Lovely, Chem. Rev., 2008, 108, 3223-3238.
27. J. Mehara, B. T. Watson, A. Noonikara-Poyil, A. O. Zacharias, J. Roithová and H. V. R. Dias, Chem. Eur. J., 2022, 28, e202103984.
28. M. S. Nechaev, V. M. Rayón and G. Frenking, J. Phys. Chem., A, 2004, 108, 3134-3142.
29. K. Klimovica, K. Kirschbaum and O. Daugulis, Organometallics, 2016, 35, 2938-2943.
30. I. Krossing and A. Reisinger, Angew. Chem. Int. Ed., 2003, 42, 5725-5728.
31. H. V. R. Dias and J. Wu, Eur. J. Inorg. Chem., 2008, 2008, 509-522.
32. Y. Yin, Z. Zhang, C. Xu, H. Wu, L. Shi, S. Wang, X. Xu, A. Yuan, S. Wang and H. Sun, ACS Sustainable Chem. Eng., 2020, 8, 823-830.
33. R. B. Eldridge, Ind. Eng. Chem. Res., 1993, 32, 2208-2212.
34. M. G. Cowan, W. M. McDanel, H. H. Funke, Y. Kohno, D. L. Gin and R. D. Noble, Angew. Chem. Int. Ed., 2015, 54, 5740-5743.
35. K. A. Reid and D. C. Powers, Chem. Commun., 2021, 57, 4993-5003.
36. F. M. Chadwick, T. Kramer, T. Gutmann, N. H. Rees, A. L. Thompson, A. J. Edwards, G. Buntkowsky, S. A. Macgregor and A. S. Weller, J. Am. Chem. Soc., 2016, 138, 13369-13378.
37. F. M. Chadwick, A. I. McKay, A. J. Martinez-Martinez, N. H. Rees, T. Krämer, S. A. Macgregor and A. S. Weller, Chem. Sci., 2017, 8, 6014-6029.
38. J. C. Goodall, M. A. Sajjad, E. A. Thompson, S. J. Page, A. M. Kerrigan, H. T. Jenkins, J. M. Lynam, S. A. Macgregor and A. S. Weller, Chem. Commun., 2023, 59, 10749-10752.
39. E. Y. Slobodyanyuk, O. S. Artamonov, O. V. Shishkin and P. K. Mykhailiuk, Eur. J. Org. Chem., 2014, 2014, 2487-2495.
40. A. A. Mohamed, L. M. Pérez and J. P. Fackler, Inorg. Chim. Acta, 2005, 358, 1657-1662.
41. H. V. R. Dias and H. V. K. Diyabalanage, Polyhedron, 2006, 25, 1655-1661.
42. Y. Morishima, D. J. Young and K. Fujisawa, Dalton Trans., 2014, 43, 15915-15928.
43. H. V. R. Dias, Z. Wang and W. Jin, Inorg. Chem., 1997, 36, 6205-6215.
44. H. V. R. Dias and X. Wang, Dalton Trans., 2005, DOI: 10.1039/B510660E, 2985-2987.
45. H. A. Chiong and O. Daugulis, Organometallics, 2006, 25, 4054-4057.
46. H. V. R. Dias, J. Wu, X. Wang and K. Rangan, Inorg. Chem., 2007, 46, 1960-1962.
47. A. Reisinger, N. Trapp and I. Krossing, Organometallics, 2007, 26, 2096-2105.
48. S. Uchida, R. Kawamoto, H. Tagami, Y. Nakagawa and N. Mizuno, J. Am. Chem. Soc., 2008, 130, 12370-12376.
49. X. Kou and H. V. R. Dias, Dalton Trans., 2009, DOI: 10.1039/B910805J, 7529-7536.
50. A. Reisinger, N. Trapp, C. Knapp, D. Himmel, F. Breher, H. Rüegger and I. Krossing, Chem. Eur. J., 2009, 15, 9505-9520.
51. H. V. R. Dias and J. Wu, Organometallics, 2012, 31, 1511-1517.
52. N. B. Jayaratna, I. I. Gerus, R. V. Mironets, P. K. Mykhailiuk, M. Yousufuddin and H. V. R. Dias, Inorg. Chem., 2013, 52, 1691-1693.
53. M. Stricker, B. Oelkers, C. P. Rosenau and J. Sundermeyer, Chem. Eur. J., 2013, 19, 1042-1057.
54. S. G. Ridlen, J. Wu, N. V. Kulkarni and H. V. R. Dias, Eur. J. Inorg. Chem., 2016, 2016, 2573-2580.
55. M. Navarro, J. Miranda-Pizarro, J. J. Moreno, C. Navarro-Gilabert, I. Fernández and J. Campos, Chem. Commun., 2021, 57, 9280-9283.
56. M. Vanga, A. Muñoz-Castro and H. V. R. Dias, Dalton Trans., 2022, 51, 1308-1312.
57. B. T. Watson, M. Vanga, A. Noonikara-Poyil, A. Muñoz-Castro and H. V. R. Dias, Inorg. Chem., 2023, 62, 1636-1648.
58. M. Fianchini, C. F. Campana, B. Chilukuri, T. R. Cundari, V. Petricek and H. V. R. Dias, Organometallics, 2013, 32, 3034-3041.
59. H. V. R. Dias and M. Fianchini, Angew. Chem. Int. Ed., 2007, 46, 2188-2191.
60. I. I. Gerus, R. X. Mironetz, I. S. Kondratov, A. V. Bezdudny, Y. V. Dmytriv, O. V. Shishkin, V. S. Starova, O. A. Zaporozhets, A. A. Tolmachev and P. K. Mykhailiuk, J. Org. Chem., 2012, 77, 47-56.
61. H. Schmidbaur and A. Schier, Angew. Chem. Int. Ed., 2015, 54, 746-784.
62. C. V. Hettiarachchi, M. A. Rawashdeh-Omary, D. Korir, J. Kohistani, M. Yousufuddin and H. V. R. Dias, Inorg. Chem., 2013, 52, 13576-13583.
63. L. Krause, R. Herbst-Irmer, G. M. Sheldrick and D. Stalke, J. Appl. Crystallogr., 2015, 48, 3-10.
64. G. M. Sheldrick, Acta Crystallogr. A, Found. Adv., 2015, 71, 3-8.
65. G. M. Sheldrick, Acta Crystallogr. C, Struct. Chem., 2015, 71, 3-8.
66. O. V. Dolomanov, L. J. Bourhis, R. J. Gildea, J. A. K. Howard and H. Puschmann, J. Appl. Crystallogr., 2009, 42, 339-341.
67. P. J. Chupas, K. W. Chapman, C. Kurtz, J. C. Hanson, P. L. Lee and C. P. Grey, J. Appl. Crystallogr., 2008, 41, 822-824.
68. B. H. Toby and R. B. Von Dreele, J. Appl. Crystallogr., 2013, 46, 544-549.
69. A. Hammersley, S. Svensson, M. Hanfland, A. Fitch and D. Hausermann, High Press. Res., 1996, 14, 235-248.
70. A. A. Coelho, Journal, 2016, DOI: http://www.topas-academic.net.
71. S. ADF 2019, Theoretical Chemistry, Vrije Universiteit, Amsterdam, The Netherlands, Journal, http://www.scm.com.
72. A. D. Becke, Phys. Rev. A, 1988, 38, 3098-3100.
73. S. Grimme, WIREs Computational Molecular Science, 2011, 1, 211-228.
74. L. Versluis and T. Ziegler, J. Chem. Phys., 1988, 88, 322-328.
75. T. Ziegler and A. Rauk, Inorg. Chem., 1979, 18, 1558-1565.

1. A composition comprising an alkene and a compound having Formula I: 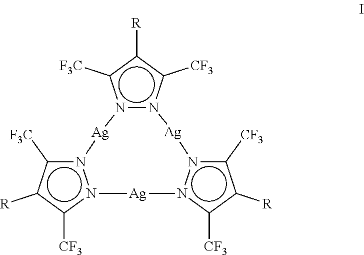
wherein each R is independently chosen from H, CF₃, and Br.

2. The composition of claim 1, wherein the alkene is ethene, propene, butene, or a mixture thereof.

3. The composition of claim 1, further comprising an alkane, wherein the alkane comprises one or more of ethane, propane, and butane.

4. The composition of claim 1, wherein Formula I exhibits a reversible transformation to Formula II: 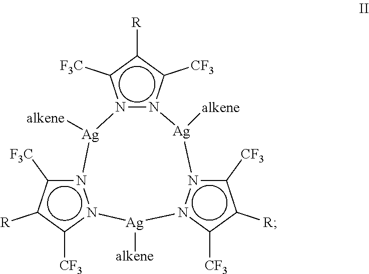
and/or Formula III: 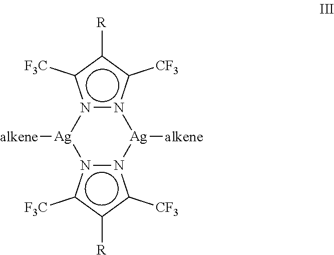
wherein each R is independently chosen from H, CF₃, and Br, and wherein the alkene of the composition is a same alkene of Formula II and Formula III.

5. The composition of claim 4, wherein Formula II is formed by an alkene adsorption on Formula I when R is H.

6. The composition of claim 4, wherein Formula III is formed by an alkene adsorption on Formula I when R is CF₃ or Br.

7. The composition of claim 4, wherein an alkene desorption transforms Formula II to Formula I.

8. The composition of claim 4, wherein an alkene desorption transforms Formula III to Formula I.

9. The composition of claim 4, wherein the reversible transformation is an endothermic structural rearrangement of Formula 1.

10. The composition of claim 4, wherein the reversible transformation is a solid-state transformation.

11. A method of separating an alkene from a mixture, the method comprising:
contacting the mixture to a compound having Formula I: 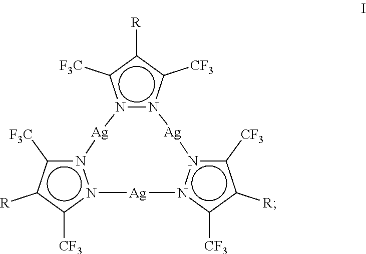
to form a complex having Formula II: 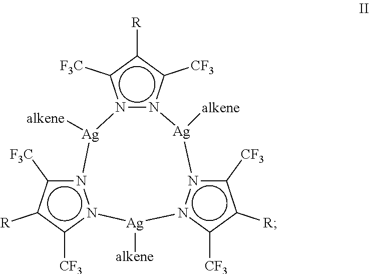
and/or Formula III: 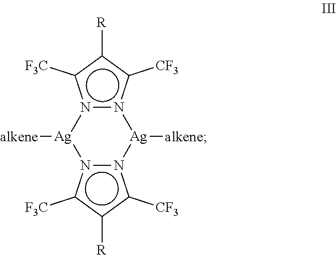
wherein each R is independently chosen from H, CF₃, and Br.

12. The method of claim 11, wherein the mixture is contacted with the compound having Formula I at a pressure below a partial pressure of the alkene.

13. The method of claim 11, wherein the mixture is contacted with the compound having Formula I at a temperature from −75° C. to 25° C.

14. The method of claim 11, wherein the mixture is contacted with the compound having Formula I at a pressure from ambient pressure to 100 kPa.

15. The method of claim 11, wherein the mixture is contacted with the compound having Formula I at a pressure from 100 kPa to 6,000 kPa.

16. The method of claim 15, wherein the mixture is contacted with the compound having Formula I at a pressure from 300 kPa to 1,000 kPa.

17. The method of claim 11, further comprising reducing pressure to ambient pressure or below after forming the complex having Formula II and/or Formula III and collecting the alkene.

18. The method of claim 11, further comprising increasing temperature after forming the complex having Formula II and/or Formula III and collecting the alkene.

19. The method of claim 11, wherein the mixture is contacted with the compound having Formula I in the presence of a solvent.

20. The method of claim 11, wherein the mixture is a hydrocarbon feed gas mixture.
